(19) United States
(12) Patent Application Publication
(10) Pub. No.: US 2008/0052009 A1
(43) Pub. Date: Feb. 28, 2008

(54) METHOD FOR DECONVOLVING SINGLE-MOLECULE INTENSITY DISTRIBUTIONS FOR QUANTITATIVE BIOLOGICAL MEASUREMENTS

(75) Inventors: Daniel T. Chiu, Seattle, WA (US); Sarah Ann Nielsen, Seattle, WA (US); Bryant S. Fujimoto, Seattle, WA (US); Christopher Lee Kuyper, Seattle, WA (US); Jason S. Kuo, Seattle, WA (US); Sandra M. Bajjalieh, Seattle, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: WASHINGTON, UNIVERSITY OF, Seattle, WA (US)

(21) Appl. No.: 11/844,250

(22) Filed: Aug. 23, 2007

Related U.S. Application Data
(60) Provisional application No. 60/823,342, filed on Aug. 23, 2006.

(51) Int. Cl. G01N 33/48

(52) U.S. Cl. 702/21

(57) ABSTRACT

A method for quantifying fluorescent puncta comprising acquiring at least one first intensity distribution comprising fluorescence intensity values from a plurality of first fluorescent puncta; acquiring at least one second intensity distribution comprising fluorescence intensity values from a plurality of second fluorescent puncta, wherein each second fluorescent puncta has a determined number of fluorescent emitters; determining the relationship between the first and second intensity distributions; and fitting the second intensity distribution to the first intensity distribution to provide a count and distribution of the number of fluorescent emitters within the first fluorescent puncta.

### CROSS-REFERENCE TO RELATED APPLICATION

This application claims the benefit of U.S. Provisional Application No. 60/823,342, filed Aug. 23, 2006, which application is incorporated herein by reference in its entirety.

### STATEMENT OF GOVERNMENT LICENSE RIGHTS

This invention was made with Government support under Contract No. RO1 NS052637, awarded by the National Institutes of Health. The Government has certain rights in the invention.

### BACKGROUND OF THE INVENTION

The need for quantitative tools in biology is growing as the information drawn from biological measurements becomes more precise. Fluorescence microscopy images of cells often contain puncta, in which the fluorescent molecules of interest are spatially concentrated. The ability to count both the absolute number and the variation in the number of molecules present in these puncta, or regions of interest (ROIs), will advance quantitative biological measurements. Knowing the concentration of proteins within a ROI provides the opportunity to study a biological system at a level of detail that is inaccessible to traditional biochemical techniques.

To measure the concentration of fluorescently labeled proteins in a given ROI, calibration typically needs to be performed for each fluorescent species. The method of calibration will limit the precision of the measurement, and often the dynamic range of the technique as well. Recently developed calibration techniques are tailored for high copy-number proteins and they only provide average values rather than exact numbers, owing to the presence of fluorescence intensity distributions inherent in the measurements. These methods for measuring protein number use the average fluorescence intensity obtained during calibration. As a result, the presence of intensity distributions about the mean value complicates calibration, which can lead to large uncertainties in the actual number of the measured fluorescent proteins.

These uncertainties are especially acute when dealing with proteins that are of low to intermediate abundance (a few to tens of copy numbers). To address this problem, previously reported counting methods can record a single molecule intensity value that is used to divide into greater intensity values, which correspond to fluorescent puncta or groups of molecules that contain multiples of the single molecule and thus can be counted to determine the number of molecules present in each puncta or group. Similarly, intensities from a population of single molecules that were approximated with Gaussian distributions were recorded to determine the mean intensity values and widths. This method was capable of counting molecules present within each fluorescent puncta and determining the probability distribution defining the numbers of fluorescein-biotin conjugates bound to streptavidin. Unfortunately, these approaches are limited to counting low number of molecules (<4) and also preclude the use of probes that have a broad initial fluorescence intensity distribution, such as dye-tagged antibodies. Recent measurements of local protein concentration are based on fusion with fluorescent proteins, which guarantees one fluorophore per protein. In contrast, however, dye-tagged antibodies are labeled via primary amines and the number of fluorophores per antibody can vary significantly. Therefore, dye-tagged antibodies can give rise to even broader fluorescence intensity distributions than single green fluorescent proteins (GFPs), which can lead to an even greater error in the estimation of the actual number of proteins present and thus precludes their use in protein counting. Although such inherent intensity distributions can complicate calibration, they can also provide a wealth of information about the system. Moreover, these distributions are dependent upon both the sample and experimental conditions. Thus, methods that extract this useful information and require no prior knowledge of the sample or experimental conditions will offer novel ways to overcome the current problems facing quantitative biological experiments.

### SUMMARY OF THE INVENTION

In one aspect, the present invention provide a method for quantifying fluorescent puncta. In one embodiment, the method of the invention includes the steps of:

(a) acquiring at least one first intensity distribution, wherein the first intensity distribution comprises fluorescence intensity values from a plurality of first fluorescent puncta, and wherein each first fluorescent puncta comprises at least one fluorescent emitter;

(b) acquiring at least one second intensity distribution, wherein the second intensity distribution comprises fluorescence intensity values from a plurality of second fluorescent puncta, wherein each second fluorescent puncta has a determined or known number of fluorescent emitters;

(c) determining the relationship between the first and second intensity distributions; and

(d) fitting the second intensity distribution to the first intensity distribution to provide a count and distribution of the number of fluorescent emitters within the first fluorescent puncta.

In one embodiment, the first intensity distribution is a normal distribution, a lognormal distribution, or a combination thereof. In one embodiment, the second intensity distribution is a normal distribution, a lognormal distribution, or a combination thereof.

In one embodiment, determining the relationship between the first and second intensity distributions comprises scale analysis. In one embodiment, determining the relationship between the first and second intensity distributions comprises shape analysis. In one embodiment, determining the relationship between the first and second intensity distributions comprises statistical analysis. In one embodiment, determining the relationship between the first and second intensity distributions comprises analytical calibration analysis.

In one embodiment, the relationship between the first and second intensity distributions is multiplicative, additive, or a combination thereof.

In one embodiment, fitting the second intensity distribution to the first intensity distribution comprises a closeness-of-fit calculation.

The method of the invention can be used to quantify fluorescence puncta, which include at least one fluorescent emitter Representative emitters include fluorescent molecules, antibodies, antibody fragments, proteins, peptides, subcellular organelles, subcellular structures, subcellular compartments, protein complexes, nanoparticles, and quantum dots. Fluorescent puncta can include a fluorescent emitter in contact with a biological entity. Representative biological entities include synaptic vesicles, liposomes, biological cells, organelles, proteins, DNAs, signaling complexes, or antibodies. The fluorescent puncta can be located in a cell or on a cell, be immobilized, or in flow.

Fluorescence emission from the fluorescent puncta can result from single photon excitation, two-photon excitation, multi-photon excitation, chemiluminescent excitation, or electroluminescent excitation. Fluorescence intensity values can be acquired by any one of a variety of imaging systems. Representative systems include wide area epifluorescence microscopes, confocal microscopes, scanning confocal microscopes, total internal reflection fluorescence microscopes, two-photon microscopes, scanning two-photon microscopes, and stimulated emission depletion (STED) microscopes.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 depicts fluorescence intensity distributions of single molecules and particles. FIG. 1A is an image of single Alexa 488 dye molecules; the right panel shows each molecule being circled automatically by the imaging software to define a region of interest. FIG. 1B are probability versus intensity graphs illustrating intensity distributions of single Alexa 488 molecules; FIG. 1C are probability versus intensity graphs illustrating single goat anti-mouse IgG molecules labeled with multiple Alexa 488 molecules; and FIG. 1D are probability versus intensity graphs illustrating single synaptic vesicles tagged with anti-synaptic vesicle protein 2 primary antibody and Alexa 488 labeled goat anti-mouse secondary antibody. Corresponding normal and lognormal best fits accompany each measured intensity distribution. Laser power was 88 μW after the objective; camera exposure time was 300 ms exposure.

FIG. 2 shows effects of multiplying the initial distribution. FIG. 2A compares probability plots of theoretical intensity distributions after multiplication of the initial distribution (i.e., MD process) by a factor of 1-5 (c=1-5). FIG. 2B compares corresponding lognormal cumulative probability plots for c=1-5.

FIG. 3 shows effects of randomly adding the initial distribution. FIG. 3A compares probability plots of theoretical intensity distributions after randomly adding (i.e., RA process) the initial distribution 1-5 times (c=1-5). FIG. 3B compares corresponding lognormal cumulative probability plots for c=1-5.

FIG. 4 shows a comparison of RA and MD basis vectors. FIG. 4A compares probability plots of the basis vectors generated by the MD (solid) and RA (dashed) process for c=2 and 3. FIG. 4B compares the ratio of σ/μ of the MD and RA basis vectors for c=1-8; a decrease in the ratio for RA indicates a decrease in the relative width of the distribution.

FIG. 5 shows best-fit results for simulated distribution 3RA (see Tables 1 and 2; a Case I example) fitted with MD (FIG. 5A) and RA (FIG. 5B) basis vectors: vertical bar is simulated data (yᵢ); dashed line is the best fit result (y′ᵢ); the solid lines are a_{c}ρ_{c} (x) (see Table 2), and the dotted line is the residuals of the fit (y′ᵢ(x)−yᵢ(x)).

FIG. 6 shows best-fit results for simulated distribution 5MD (see Tables 1 and 3; a Case II example) fitted with MD (FIG. 6A) and RA (FIG. 6B) basis vectors: vertical bar is simulated data (yᵢ); dashed line is the best fit result (y′ᵢ); the solid lines are a_{c}ρ_{c} (x) (see Table 3); and the dotted line is the residuals of the fit (y′ᵢ(x)−yᵢ(x)).

FIG. 7 depicts a distribution of coefficients from MD and RA fits of 4MD (FIG. 7A) and 8MD (FIG. 7B) (see Tables 1 and 4; a Case III example).

FIG. 8 illustrates single-molecule measurements of the binding of Alexa 488-tagged biocytin to avidin. FIG. 8A depicts the results of applying the MD deconvolution analysis. FIG. 8B depicts the results of applying the RA analysis. FIG. 8C is a histogram showing the percentage of avidin with c biocytins obtained from the MD and RA analysis.

### DETAILED DESCRIPTION OF THE INVENTION

In one aspect, the present invention provide a method for quantifying fluorescent puncta. As used herein, the term “quantifying fluorescent puncta” refers to determining the number (count) of fluorescent emitters (or units) in fluorescent puncta. In one embodiment, the method provides the number of fluorescent emitters in the fluorescent puncta. In one embodiment, the method provides a number distribution of fluorescent emitters in the fluorescent puncta.

In the method, two fluorescence intensity distributions are acquired. The first fluorescence intensity distribution corresponds to the fluorescent puncta having an undetermined number of fluorescent emitters (ROI intensity distribution). The second fluorescence intensity distribution corresponds to the fluorescent puncta having a known or determined number of fluorescent emitters (single-molecule intensity distribution).

Once the two fluorescence intensity distributions are acquired, the relationship between the two distributions is determined. The relationship between the first and second intensity distributions can be determined by any one of a variety of methods. The relationship between the first and second intensity distributions can be determined by, for example, scale analysis, shape analysis, statistical analysis, or analytical calibration analysis. The relationship between the first and second intensity distributions may be variable. The relationship may be, for example, multiplicative, additive, or some combination of multiplicative and additive.

In the method, the second intensity distribution is used to fit the first intensity distribution to determine the number of fluorescent emitters in the fluorescent puncta (e.g., ROI). Fitting the second intensity distribution to the first intensity distribution can be performed by any one of a variety of methods. In one embodiment, fitting the second intensity distribution to the first intensity distribution utilizes a closeness-of-fit calculation.

In one embodiment, the method of the invention includes the steps of:

(a) acquiring at least one first intensity distribution, wherein the first intensity distribution comprises fluorescence intensity values from a plurality of first fluorescent puncta, and wherein each first fluorescent puncta comprises at least one fluorescent emitter;

(b) acquiring at least one second intensity distribution, wherein the second intensity distribution comprises fluorescence intensity values from a plurality of second fluorescent puncta, wherein each second fluorescent puncta has a determined or known number of fluorescent emitters;

(c) determining the relationship between the first and second intensity distributions; and

(d) fitting the second intensity distribution with at least one integer ratio of the first intensity distribution to provide a count and distribution of the number of fluorescent emitters within the first fluorescent puncta.

In one embodiment, the first intensity distribution is a normal distribution, a lognormal distribution, or a combination thereof. In one embodiment, the second intensity distribution is a normal distribution, a lognormal distribution, or a combination thereof.

In one embodiment, determining the relationship between the first and second intensity distributions comprises scale analysis. In one embodiment, determining the relationship between the first and second intensity distributions comprises shape analysis. In one embodiment, determining the relationship between the first and second intensity distributions comprises statistical analysis. In one embodiment, determining the relationship between the first and second intensity distributions comprises analytical calibration analysis.

In one embodiment, the relationship between the first and second intensity distributions is multiplicative, additive, or a combination thereof.

In one embodiment, fitting the second intensity distribution to the first intensity distribution comprises a closeness-of-fit calculation.

The method of the invention can be used to quantify fluorescence puncta, which include at least one fluorescent emitter The fluorescent element or emitter can be any suitable fluorescence emitter. Representative emitters include fluorescent molecules, antibodies, antibody fragments, proteins, peptides, subcellular organelles, subcellular structures, subcellular compartments, protein complexes, nanoparticles, and quantum dots.

The fluorescent puncta can include a fluorescent emitter in contact with a biological entity. Representative biological entities include synaptic vesicles, liposomes, biological cells, organelles, proteins, DNAs, signaling complexes, or antibodies. The fluorescent puncta can be located in a cell or on a cell, be immobilized, or in flow.

Fluorescence from the fluorescent puncta quantified by the method of the invention can result from single photon excitation, two-photon excitation, multi-photon excitation, chemiluminescent excitation, or electroluminescent excitation.

As noted above, the invention provides a method for deconvolving intensity distributions that utilizes not only the average intensity value to calibrate fluorescence intensities, but also the shape of the intensity distribution about the mean value to deconvolve the measured intensities. The method is effective over a wide dynamic range for counting proteins or other molecules present in a single copy to tens of copies.

To quantify the number of fluorescent molecules present in a sample of ROIs, the relationship between the single-molecule intensity distribution and the single-ROI intensity distribution need to be understood. Once the relationship is understood, the single-molecule intensity distribution can be used to generate basis vectors to fit the ROI intensity distribution and thus the measure number of fluorescent molecules present in the ROIs. The single-molecule and ROI distributions have either a multiplicative (MD) or additive (RA) relationship. For example, a collection of puncta that each has three molecules of interest could generate an intensity distribution that either reflects each single-molecule calibration value multiplied by three, or a collection of three randomly-selected calibration values added together.

In most cases, the shape (i.e., whether the distribution is normal or lognormal) of the single-ROI distribution serves as a good indicator as to whether the two distributions are related additively or multiplicatively. In addition to using the shape of the ROI distribution to determine the relationship between the two distributions, the goodness of fit (reduced chi-squared) can be used as a statistical criteria to determine the correct relationship.

In the following discussion, the distribution used for calibration is referred to as the single-molecule distribution or single-fluorophore distribution. Calibration distributions are composed of the fluorescent units to be counted in ROIs. For example, single antibodies labeled with multiple fluorophores generate calibration curves for antibody-labeled samples, whereas single synaptic vesicles (e.g., labeled with FM dyes) would be used to generate a calibration curve to count the number of vesicles per synapse in cultured neurons or tissue slices. It is important to note that any number of cellular structures, molecules, or fluorescent emitters can be counted. In addition, the counting of the method of the invention can be performed using a wide range of microscopy techniques and modes of microscopy imaging, including wide-area epifluorescence, scanning confocal, total internal reflection fluorescence, scanning two-photon, scanning multiphoton, coherent anti-stokes Raman scattering (CARS), second harmonic generation, and stimulated emission depletion (STED) microscopy.

Fluorescence Intensity Distributions

The distribution of fluorescence emission intensities of a set of ROIs is denoted ρ(x), where ρ(x)dx is the probability that a ROI in the set will exhibit an intensity between x and x+dx. For the case where the set is not monodisperse, each of the ROIs can contain different numbers of fluorophores, and ρ(x) can be written as a weighted sum of intensity distributions:

$$ρ ( x ) = \frac{1}{N} {\sum }_{c = 1}^{M} {A}_{c} {ρ}_{c} ( x ) \qquad ( 1 )$$

where ρ_{c} (x)dx is the probability that a ROI with c fluorophores will exhibit an intensity emission between x and x+dx; the coefficient A_{c} is the actual number of ROIs in the set with c fluorophores; N is the total number of ROIs in the set, and M is the largest number of fluorophores contained by any ROI in the set. The actual intensity data will be a set of values that can be converted into a histogram with elements yᵢ, the number of ROIs whose emission intensity falls into the ith bin. The ρ_{c}(x) then can be expressed as normalized histograms with elements f_{c} (i) and these will be referred to as basis vectors. These basis vectors will be used later for fitting the measured intensity distributions (see Data Fitting section).

With these changes we can write

$${y}_{i} = {\sum }_{c = 1}^{M} {A}_{c} {f}_{c} ( i ) \\ N = {\sum }_{i = 1}^{L} {y}_{i} \qquad ( 2 )$$

where L is the number of bins in the histograms. Because the basis vectors are normalized,

$$1 = {\sum }_{i = 1}^{L} {f}_{c} ( i ) \\ N = {\sum }_{c = 1}^{M} {A}_{c} \qquad ( 3 )$$

the intensity distribution for single fluorophores, ρ₁(x), can be obtained by fitting the set of measured single fluorophore intensities to an appropriate functional form. This operation prevents any noise in the measured set of single fluorophore intensities from being propagated into the basis vectors where they could increase the uncertainty in the results of data fitting. On the other hand, if the single-fluorophore intensity distribution cannot be described by a suitable function form, then the set of single fluorophore intensities can be used directly to form the basis vectors. The ρ_{c}(x) (or f_{c}(i) ) will be obtained from measurements of the single fluorophore intensity distribution ρ₁(x) (or f₁(i) ) by procedures described below.
Normal Distribution of Fluorescence Intensities

Properties of normal distribution. The normal distribution, which is defined for −∞<x<+∞ is

$$ρ ( x ) = \frac{1}{\sqrt{2 π {σ}^{2}}} exp ( \frac{- {( x - μ )}^{2}}{2 {σ}^{2}} ) \qquad ( 4 )$$

where μ is the mean and σ is the standard deviation for the distribution. By the central limit theorem, a random variable, which is itself the sum of J independent random variables drawn from identical distribution functions, will be normally distributed for sufficiently large J. What constitutes sufficiently large depends on the shape of the parent distribution function.

For a ROI with multiple fluorophores, if the intensities of the individual fluorophores can be regarded as independent of each other with identical intensity distributions, then we would expect for a monodisperse system where every ROI contains exactly c fluorophores that the intensity distribution will be normally distributed for sufficiently large c.

Possible sources of a distribution in intensity for which the fluorophores within a single ROI would be regarded as independent of each other include polarization effects and the random orientation of the fluorophore. The fluorescence intensity of a dipole excited by an evanescent wave in total-internal-reflection-fluorescence (TIRF) microscopy, for example, depends sensitively on the orientation of the dipole moment. This mechanism of combining emission intensities from fluorophores in a single ROI shall be denoted Random Addition or the RA process. If one observes a normal distribution in the measured fluorescence intensities from the ROIs, there is a high likelihood that the single-molecule and single-ROI distributions are related by the RA process.

RA basis vectors. The RA basis vector for ROIs with exactly c fluorophores is a convolution of c copies of ρ₁(x). If an accurate analytical form exists for ρ₁(x), the set of basis vectors can be formed by performing the convolution c times for each basis vector, and normalized histograms can be created from the resulting ρ₁(x). Otherwise, a random number generator can be used to generate a set of intensity values corresponding to ρ_{c}(x) for each value of c. For each value of c, c intensity values are selected randomly from the c=1 basis vector, which either can be a data set of single-fluorophore emission intensities or a functional representation of the single-molecule intensity distribution (ρ₁(x)). The intensities are then summed and the result added to the set of intensities. When a sufficiently large number of values have been generated (typical values range from 10,000 to 100,000) a normalized histogram is made from the values. The elements of the resulting basis vectors shall be denoted f_{c}^{A}(i).

Lognormal Distribution of Fluorescence Intensities

Properties of lognormal distribution. The lognormal distribution, which is defined for 0<x<+∞ is

$${ρ}^{*} ( x ) = \frac{1}{x \sqrt{2 π {σ}^{* 2}}} exp ( \frac{- {( ln ( x ) - {μ}^{*} )}^{2}}{2 {σ}^{* 2}} ) \qquad ( 5 )$$

where μ* is the scale parameter and σ* is the shape parameter of the distribution. The average value of x for the lognormal distribution is
<x>=x₀e^{σ}^{²}^{/2} (6)
where ln(x₀)=μ*. A random variable, which is itself the product of J independent random variables drawn from identical parent distribution functions, will exhibit a lognormal distribution for sufficiently large J. Limpert, E., et al, “Log-Normal Distributions Across the Sciences: Keys and Clues,” Bioscience 51:341, 2001.

An example of how such a random process might arise in fluorescence intensity measurements is the modest defocusing that can occur when collecting a fluorescence image. The defocusing results in decreased collection efficiency for certain ROIs, which is the equivalent of multiplying the intensity that would have been observed from that ROI by a number less than 1. Another example is the slight difference in the distance of the fluorophores from the glass/water interface owing to irregularities of the glass surface, which can affect significantly the collected fluorescence intensity in TIRF microscopy. So long as these effects are uncorrelated with the number of fluorophores in the ROIs, then this effect will appear as if the emission intensities are multiplied by a random number. Other processes that would be manifested as multiplicative factors include variation in the pixel quantum efficiency in the CCD camera, dirt and aberrations in the optics, and any spatial variation in the intensity of the illuminating evanescent wave. All of these things will combine multiplicatively to produce different excitation/collection efficiencies for different locations in the image. It is not required that any of one these effects be large, but they might combine to produce a lognormal distribution. This mechanism of combining emission intensities from fluorophores in a single ROI shall be denoted the Multiplied Distribution or the MD process. If one observes a lognormal distribution in the measured fluorescence intensities from the ROIs, then it is most likely that the single-molecule and single-ROI distributions are related by the MD process.

MD basis vectors. The basis vectors for the MD process can be obtained from the single fluorophore distribution function, ρ₁(x). Since the location of any given percentile of the distribution function ρ₁(x) increases by a factor of c in the MD process, ρ_{c}(x) can be obtained by replacing every occurrence of x in ρ₁(x)dx by (x/c), so that
ρ_{c}(x)dx=ρ₁(x/c)d(x/c) (7)
where ρ_{c}(x)dx is the probability that a ROI with c fluorophores will exhibit an emission intensity between x and x+dx. ρ_{c}(x) is converted into the normalized histogram, f_{c}^{M}(i), which is the basis vector for c fluorophores in the ROI for the MD case. If a data set of emission intensities for the single fluorophores, rather than a function, is being used for ρ₁(x), then the f_{c}^{M}(i) are obtained by multiplying each intensity in the data set by c and then forming a normalize histogram from the resulting set of intensity values.
Data Fitting

The histogram of intensity distribution is modeled by

$${y}_{i}^{′} = {\sum }_{c = 1}^{M} {a}_{c} {f}_{c} ( i ) \\ {N}^{′} = {\sum }_{c = 1}^{M} {a}_{c} \qquad ( 8 )$$

where the coefficients a_{c} are the adjustable parameters that represent an estimate of the number of ROIs containing c fluorophores, y′ᵢ is the model's estimate of yᵢ, and N′ is the estimate of N, the number of ROIs in the set. For fitting a_{c} is constrained to be positive a_{c}=|a_{c}|. The absolute value of the coefficients is used to simplify the fitting procedure, because this is simpler than restraining the fit to consider only physically reasonable (i.e., positive) values of the coefficients.

A fitting algorithm is used to minimize χ², which for our purposes is defined as

$${χ}^{2} = {\sum }_{i = 1}^{L} \frac{{( {y}_{i} - {y}_{i}^{′} )}^{2}}{{σ}_{i}^{2}} + {α ( N - {N}^{′} )}^{2} = {\sum }_{i = 1}^{L} \frac{( {y}_{i} - {y}_{i}^{′} )}{{y}_{i}} + {α ( N - {N}^{′} )}^{2} \\ {χ}_{v}^{2} = \frac{{χ}^{2}}{( L - M )} \qquad ( 9 )$$

where σᵢ² is the variance associated with the measurement of yᵢ. (L−M), the difference between the number of bins and the number of variable parameters is called the number of degrees of freedom for the fit, and χᵥ² is the reduced chi-squared for the fit. Bevington, P. R., “Data Reduction and Error Analysis for the Physical Sciences,” McGraw-Hill, New York, 1969. a is a small positive parameter that penalizes the fit if N′ deviates from the actual number of events in the data set. Typical values of α that are tried during our fits ranged from 10⁻⁶ to 10⁻². For most of our fits, values of α<10⁻³ appeared to have little effect on N′. For most fits, using α=10⁻³ was sufficient to ensure that N′ was within 1% of N. For a few fits, a larger value of α was necessary to ensure that N′ was within 1% of N, and for those the results reported below are for fits with α=10⁻².

The error in the intensity distributions was approximated by setting ρᵢ²=yᵢ. The range of bins included in the fit was chosen to be large enough to include all bins that have any intensity values from either the intensity distribution being fitted or from any of the basis vectors being used. The fitting program varies the coefficients, a_{c} in equation (8), in order to minimize χ². The best fit values of the a_{c} are then the model's estimates of the A_{c}, the actual number of ROIs in the data set with c fluorophores.

Reduced chi-squared. Ordinarily, it is expected that χᵥ² should be approximately 1 for a satisfactory fit. Two complications exist, however, which can affect the magnitude of χᵥ². First, the basis vectors used in the fit are an approximation to the actual intensity distribution for each value of c. That is, the χᵥ² obtained from the fit reflects errors in the measured distribution functions of both the ROIs and the single fluorophores (which are used to generate the basis vectors). This fact could result in an elevated value of χᵥ², even if the correct functional form is used for y′ᵢ and when only statistical noise is present. The second complication arises from the fact that in practice the actual number of basis vectors necessary is not known in advance. Consequently, the range of basis vectors used in equation (8) must be large enough to ensure it will encompass the range of species present in the sample. This consideration would generally result in the use of more basis vectors than there are species in the system. To reflect this fact, all the simulated distributions were fit using at least eight basis vectors, even though the simulated distributions were generated from only 1-4 basis vectors. The fitting program used the additional flexibility provided by the extra basis vectors to fit the noise in the simulated distribution, potentially reducing χᵥ² below 1.

These two complications do not cancel, and for some of the fits presented below, χᵥ² is significantly less than 1. This is a result of the flexibility provided by the extra basis vectors. More statistically reasonable values of χᵥ² are obtained when only the minimum number of basis vectors required are used in the fits.

Simulated annealing. Owing to the large number of basis vectors that might be used and the possible existence of local minima in χ₂, it is important that the search algorithm is capable of finding a global minimum. Initially the fits were performed using either the FMINSEARCH function of Matlab or the AMOEBA subroutine from Press et al., both of which employed the Nelder-Mead downhill simplex algorithm or the Levenberg-Marquardt algorithm (subroutine MRQMIN). Press, W.H., “Numerical Recipes in Fortran 77: The Art of Scientific Computing,” Cambridge University Press, Cambridge, England; Olsson, D. M., and L. S. Nelson, “Nelder-Mead Simplex Procedure for Function Minimization,” Technometrics 17:45, 1975. These algorithms will only search downhill in χ² where sets of coefficients that increase χ² are discarded.

To test if the algorithm has found a global minimum, the fitting procedure was repeated with six different initial guesses for the variable parameters in these fits. If all of the fits converge on the same set of best-fit coefficients, then it suggests the best-fit results do in fact represent a global minimum in χ². If the different initial guesses for the coefficients lead to different “best-fit” results, then these are local minima, and further tests are necessary to determine if one of them is the global minimum, or if there is another undetected local minimum that is the real global minimum. For most cases, the search results for the six different initial guesses converged, but there are a few instances when the search algorithms found a local minimum in χ².

To address this issue, the simulated annealing minimization program AMEBSA was used, (Press, W.H., “Numerical Recipes in Fortran 77: The Art of Scientific Computing,” Cambridge University Press, Cambridge, England) which appears to be more robust than the other two methods described above. Simulated annealing algorithms require that the user supply an initial temperature parameter, T, and a cooling schedule. Using a procedure similar to the Metropolis algorithm for Monte Carlo simulations, the search algorithm will always retain a move that results in a decrease in the value of χ², and also will retain a move that results in an increase in the value of χ² with probability

$$exp [ \frac{- {Δχ}^{2}}{T} ] \qquad ( 10 )$$

where Δχ²>0, is the increase in χ² associated with a particular search move. As a result, for nonzero values of T, the search algorithm will search both up and downhill in χ², which will enable the search algorithm to explore multiple minima in χ² if they exist. If there are multiple local minima, the search algorithm will spend more steps near the local minimum that has the smallest χ². Then as T is reduced, the search algorithm will progressively be confined near the local minimum with the smallest χ². Running AMEBSA with T=0 is equivalent to using the Nelder-Mead algorithm. Press, W.H., “Numerical Recipes in Fortran 77: The Art of Scientific Computing,” Cambridge University Press, Cambridge, England.

For a given set of initial guesses of the coefficients, χ² was calculated and T was set to χ²/4. The temperature was then reduced by a factor of 0.90 until T was less than 0.005 times (L−M), the number of degrees of freedom in the fit. This choice was motivated by the fact that for a good fit χᵥ²=1. From equation (9)
χ²=χ₀²(L−M) (11)
so the terminating value of T was chosen to be approximately 0.5% of what χ² would be if a satisfactory fit were to be obtained.

The simulated annealing program, AMEBSA, like the Nelder-Mead simplex method to which it is related, maintains a list of (M+1) sets of the variable parameters (no two of which are identical). This list is referred to as a simplex, with each set of parameters corresponding to a vertex of the simplex. For finite temperatures, there is no guarantee that the best set of parameters (smallest χ²) encountered during the search will always be a vertex in the simplex. Therefore, the simplex was checked each time the temperature parameter has been reduced by a factor of three. If the best set of parameters encountered during the fit is not a vertex in the simplex, the vertex in the simplex with the largest χ² is replaced by the best set of parameters encountered during the fit.

The result of this fit was assumed to lie near, but not necessarily at the global minimum in χ², and one additional run with T=0 was used to locate the global minimum. This procedure gave satisfactory results, and other possible initial and final temperatures or cooling schedules received only limited tests.

The Persistence of Lognormal Distributions in Single-Molecule Measurements

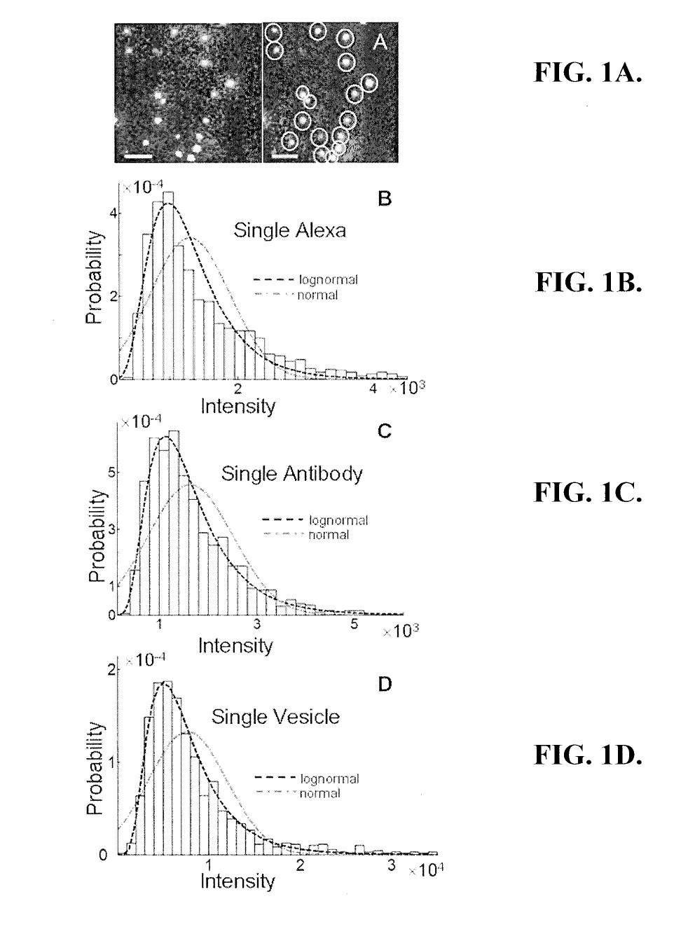

Lognormal distributions were persistently observed in the single-molecule and single-particle images. Lognormal fluorescent intensity distributions are known. Ren, X., et al., “Analysis of Human Telomerase Activity and Function By Two Color Single Molecule Coincidence Fluorescence Spectroscopy,” Journal of the American Chemical Society 128:4992, 2006; D'Agostino, R. B. and M. A. Stephens, “Goodness-of-Fit Techniques,” M. Dekker, New York, 1986. FIG. 1 shows several measured fluorescence intensity distributions, which were obtained by imaging the adsorbed molecules or vesicles on the glass coverslip illuminated with total-internal-reflection-fluorescence (TIRF) microscopy. The maximum intensity from each molecule was measured by first circling a region of interest (ROI) around the molecule, after which the imaging software automatically selected the brightest pixel from each ROI and subtracted the average background intensity from each image. FIG. 1A shows a sample image of single Alexa 488-tagged antibody molecules with sample ROI circled around each molecule. FIGS. 1B-1D plot in histogram form the background-subtracted fluorescence intensity distribution from single Alexa 488 molecules (FIG. 1B), single Alexa 488-tagged antibody molecules (FIG. 1C), and single synaptic vesicles labeled with primary (anti-synaptic vesicle protein 2) and Alexa 488-tagged secondary (goat anti-mouse) antibodies (FIGURE ID). Each distribution was fitted to both a lognormal and a normal distribution and the best fits were co-plotted with the observed intensity distribution in FIG. 1.

One might expect that the intensity, I, of any given ROI could be described as simply a sum of the intensities of the enclosed fluorophores
I=ΣIₙ (12)
where the sum runs over all the fluorophores in the ROI. That is, I is expected to be the sum of independent, identically distributed random variables. From the central limit theorem, for a sufficiently large number of fluorophores in the ROI, the distribution of intensities for the ROIs would be well approximated by a normal distribution. Yet, despite the fact that the antibodies have an average of 6 dye molecules and the vesicles have an average of 3 antibodies (and therefore an average of 18 fluorophores) attached to them, the intensity distributions for the antibodies and vesicles (FIGS. 1C and 1D) are still poorly fitted by a normal distribution. The lognormal distribution observed for the single fluorophores persists even when larger numbers of fluorophores are present in a ROI. Indeed, the measured intensity distributions of 100 nm fluorescent beads (which contain hundreds of fluorophores per bead) were found to follow also a lognormal distribution.

Two scenarios can account for the presence of lognormal distributions. The first being any variation in emission intensity between different fluorophores within the same ROI is small compared to the variations between different ROIs and thus has little effect on the observed intensity distribution. The observed intensity distribution for the ROIs would be a weighted sum of the MD distributions, with the weights corresponding to the number of occurrences of ROIs with c fluorophores. In most situations, this scenario correctly explains the persistence of lognormal distributions.

The second scenario is that the number of fluorophores in each ROI happens to be distributed in a lognormal fashion, and so the intensities that result from the application of equation (1) or (2) result in a lognormal distribution. In this case, the emission intensities of fluorophores within each ROI are independent and their distributions are identical to the distribution of emission intensities of single fluorophores. The intensity distribution for the fluorophore clusters (ROIs) would then be the result of a RA process, that is, the convolution of the single-fluorophore intensity distributions. The observed intensity distribution for the ROIs would be a weighted sum of the RA distributions. In this scenario, if each ROI contains exactly c fluorophores, then the resulting ROI distribution would be normal. A lognormal distribution is observed only when the ROIs contain a lognormal distribution in the number of fluorophores.

This second scenario is likely rare, and thus the presence of a lognormal distribution for the ROIs is a strong indication that the single-molecule and single-ROI intensities are related via the MD process. Likewise, a normal distribution is a strong indication the calibrating and ROI distributions are connected via a RA process.

Using Single-Molecule Intensities to Form MD and RA Basis Vectors The results of combining single-molecule intensities by the MD and RA processes on the observed ROI intensity distributions are illustrated by first considering the case where all the ROIs have an identical number of fluorophores, and that the distribution of single-molecule intensities is lognormal.

For a MD process, the lognormal distribution in equation (5) can be transformed using equation (7) to obtain the lognormal distribution for c fluorophores.

$$\begin{matrix}{ρ}_{c}^{*} ( x ) = \frac{1}{x \sqrt{2 {πσ}^{* 2}}} exp ( \frac{- {( ln ( x / c ) - {μ}^{*} )}^{2}}{2 {σ}^{* 2}} ) \\ = \frac{1}{x \sqrt{2 π {σ}^{* 2}}} exp ( \frac{- {( ln x - ln {cx}_{0} )}^{2}}{2 {σ}^{* 2}} )\end{matrix} \qquad ( 13 )$$

where μ*=ln(x₀) is used to show that the effect of MD on a lognormal distribution is to shift the average value of x from equation (3) to
<x>=cx₀e^{σ}^{²}^{/2} (14)

Thus this operation changed the scale (μ_{c}*=ln(cx₀)), but not the shape (σ*) of the distribution. These are the basis vectors for the MD process described earlier.

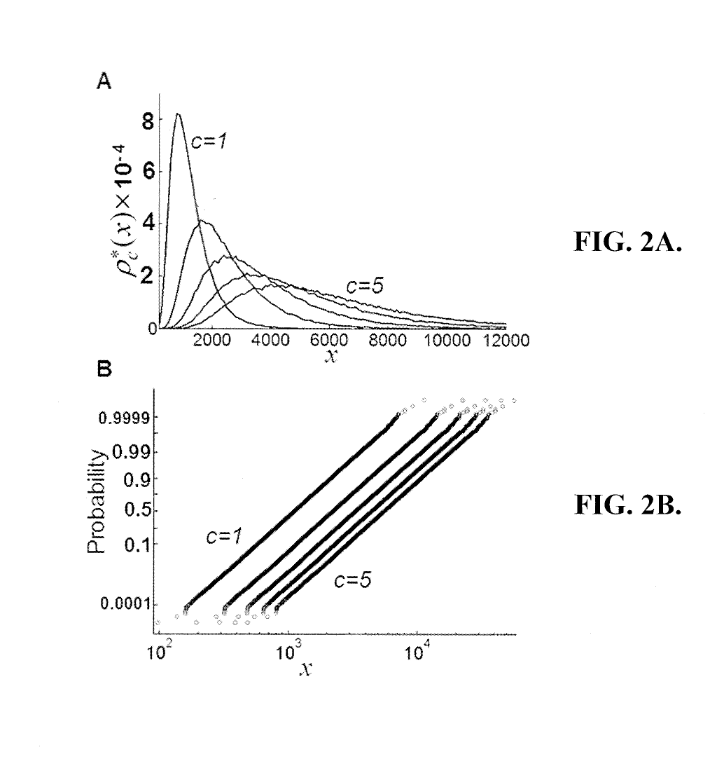

The relative width of a lognormal distribution is unchanged by MD, which is illustrated in FIG. 2A where the ρ_{c}*(x) from equation (13) with σ*=0.5 and μ*=7 is plotted for values of c increasing from 1 to 5. FIG. 2B shows an alternate method of plotting the data, where the probability is displayed. This lognormal scaled probability plot clearly illustrates that the inherent shape (which is described by the slope of the data) of distribution is not changed with increasing mean.

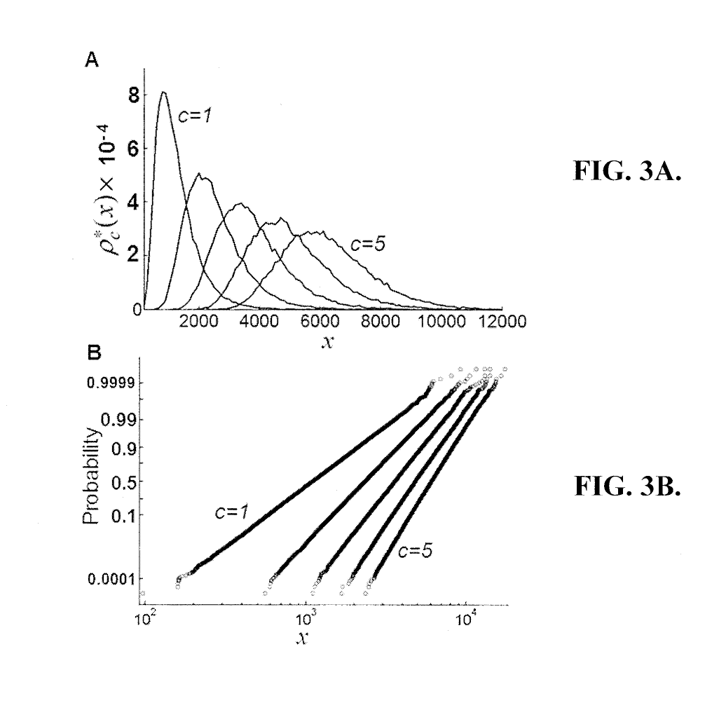

FIG. 3 shows the effect of combining fluorescence intensities by a RA process. The c=1 distribution was created by first randomly selecting values from a lognormal distribution with σ*=0.5 and μ*=7. The distribution functions for single clusters of c fluorophores are simply the basis vectors created from the RA process as described earlier. FIG. 3A plots the resulting probability functions and FIG. 3B plots the corresponding lognormal probability plots.

In this RA case, the width of the distribution grows more slowly than its mean, so the width of the distribution becomes smaller relative to its mean. This is expected, because as c increases the distribution progressively becomes better approximated by a normal distribution. FIG. 3B illustrates the change in the shape of the distribution with increasing c, as evinced by the change in the slope of the probability plot.

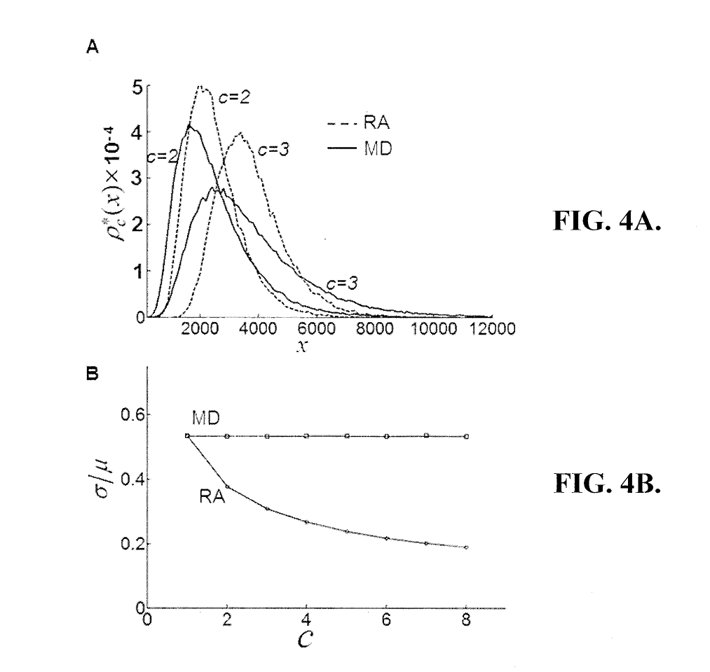

To show the difference in shape between the MD and RA generated basis vectors, FIG. 4A plots the probability densities for c=2 and 3. A measure of the shape can be obtained by calculating the relative width of each distribution. To calculate the shape and scale parameters, a lognormal distribution with σ*=0.5 and μ*=7 was sampled 100,000 times. Those values were then used as the c=l basis vector and the MD and RA processes were used to obtain basis vectors for values of c ranging from 2 to 8. The resulting σ and μ parameters were calculated and the ratio is plotted in FIG. 4B. Here the steady change in shape of the RA basis vectors away from the MD basis vectors is evident, and indicates the RA basis vectors are converging toward a normal distribution. In contrast, the MD basis vectors retain the original shape parameter. This result is expected, because the central limit theorem predicts the RA basis vectors will convert into a normal distribution for sufficiently large c. It is this change in shape that creates the possibility of being able to distinguish between a MD process and a RA process, even when there are multiple and variable number of species present in each ROI.

A more quantitative measure of shape can be obtained by calculating the skewness (γ₁) and kurtosis (γ₂) of the basis vectors as a function of c. These can be expressed in terms of μₙ, the nth moment of the distribution about the mean as:

$$\begin{matrix}{γ}_{1} = \frac{{μ}_{3}}{{μ}_{2}^{3 / 2}} \\ {γ}_{2} = \frac{{μ}_{4}}{{μ}_{2}^{2}} - 3\end{matrix} \qquad ( 15 )$$

For a normal distribution, both γ₁ and γ₂ are zero. Calculations (not shown) of γ₁ and γ₂ for the basis vectors in FIGS. 2 and 3 behave as expected; for the MD basis vectors, γ₁ and γ₂ are independent of c, and for the RA basis vectors both γ₁ and γ₂ decrease as c increases.

Simulation of Intensity Distributions

To study the differences in shape of the intensity distributions for MD and RA processes, eight sets of the coefficients, A_{c}, were first selected. A broad range of starting values simulating both monodisperse and polydisperse samples was selected. For each set, an intensity distribution was then calculated using both the MD and RA process as described below.

Generating distributions. A lognormal distribution with σ*=0.4 and μ*=6 was used as ρ₁(x). 50000 values were randomly selected from the distribution and used them to generate simulated distributions with each containing 3000 intensity values. Table 1 lists the sets of coefficients and the ratio of σ/μ for each simulated intensity distribution. For each nonzero A_{c} in a MD simulation, A_{c} values were selected at random from the set of 50000 and multiplied by c. This operation was repeated for all nonzero A_{c} in the set of coefficients. Poisson distributed noise was added to each member of the set of intensity values and the results were sorted into bins to form a histogram of the simulated intensity distribution for the MD process.

For each nonzero A_{c} in a RA simulation, c values would be selected at random from the set of 50000 and added to create an intensity value. This process was repeated A_{c} times for each nonzero A_{c} in the set of coefficients. Again Poisson distributed noise was added to the elements of the set of intensity values and the results sorted into bins to form a histogram of the simulated intensity distribution for the RA process.

TABLE 1

| | | |
|---|---|---|
| Coefficient | Simulation | σ/μ |

| Set | | Value | MD | RA |
|---|---|---|---|---|
| 1 | A₁ | 1500 | 0.56 | 0.48 |
| | A₂ | 1500 | | |
| 2 | A₂ | 1500 | 0.48 | 0.33 |
| | A₃ | 1500 | | |
| 3 | A₃ | 1500 | 0.45 | 0.26 |
| | A₄ | 1500 | | |
| 4 | A₄ | 1500 | 0.43 | 0.21 |
| | A₅ | 1500 | | |
| 5 | A₂ | 3000 | 0.43 | 0.30 |
| 6 | A₄ | 3000 | 0.43 | 0.23 |
| 7 | A₁ | 200 | 0.52 | 0.41 |
| | A₂ | 1200 | | |
| | A₃ | 1200 | | |
| | A₄ | 400 | | |
| 8 | A₃ | 200 | 0.46 | 0.26 |
| | A₄ | 1200 | | |
| | A₅ | 1200 | | |
| | A₆ | 400 | | |

Table 1 shows a list of the sets of coefficients used for generating the simulated intensity distributions for MD and RA processes; coefficients that are not listed equaled zero in that set. The distributions by the process (MD or RA) used to generate them and the number of the coefficient set as listed in the table. 3RA, for example, denotes the distribution generated using the RA basis vectors using the third set of coefficients listed in the table (i.e., with A_{c}=0, A_{c}=0, A_{c}=1500, and A_{c}=1500). Listed also are the ratios of the standard deviation to the mean (σ/μ) of the resultant distributions, which should be compared to the ratio calculated for the simulated single-fluorophore distribution that equals 0.42.

Three types of distributions. The ratio σ/μ in Table 1 serves as a measure of the relative width of each distribution, which can be compared to (σ/μ)=0.42 for the single fluorphone distribution, ρ₁(x). The results indicate there are three classes of distributions, so the simulation was categorized into three cases. Case I includes all of the simulated distributions whose relative width, (σ/μ)_{distribution}, is smaller than the relative width of ρ₁(x). All of the examples in Case I occur for distributions generated using the RA process. These distributions cannot be fitted satisfactory using MD basis vectors, and so a reduction in relative width constitutes an indicator that an RA process is occurring.

For the other two cases, (ρ/μ)_{distribution}≧(σ/μ)₁. For Case II, the fits to the two different sets of basis vectors result in significantly different values of χᵥ², in which case MD and RA processes can be distinguished and confirmed using the χᵥ² values. Case III is the scenario where the χᵥ² from both MD and RA fits are of comparable magnitude. To distinguish MD and RA processes for Case III requires either additional information about the biological system (e.g., there is an upper bound in the number of molecules within a ROI) or experimental calibration of the microscope and the measurement process.

Fitting of distributions. To fit the distributions, two sets of basis vectors (MD and RA) were generated by randomly selecting a set of 2000 values from the lognormal distribution (σ*=0.4, μ*=6), which were then used to generate the MD and RA basis vectors as described above. The set of 2000 values used to generate the basis vectors is distinct from the 50000 values used to generate the intensity distribution. The basis vectors (either f_{c}^{A}(i) or f_{c}^{M}(i) ) are used in equation (8) to model the observed intensity distribution for the ROIs.

As noted above, the inclusion of additional basis vectors in a fit beyond those used to generate the simulated distribution can result in χᵥ² that are much smaller than what might seem statistically reasonable. Several examples of this can be seen in Tables 2-4. For these cases, the fits were repeated to include only those basis vectors used to generate the distribution. The resulting value of χᵥ² all increased into a more statistically reasonable range (0.6-1.7). The larger values of χᵥ² reflect the other problem discussed earlier, namely the fact that the basis vectors are only an approximation to the parent distribution function and so χᵥ² reflects errors present in both sets.

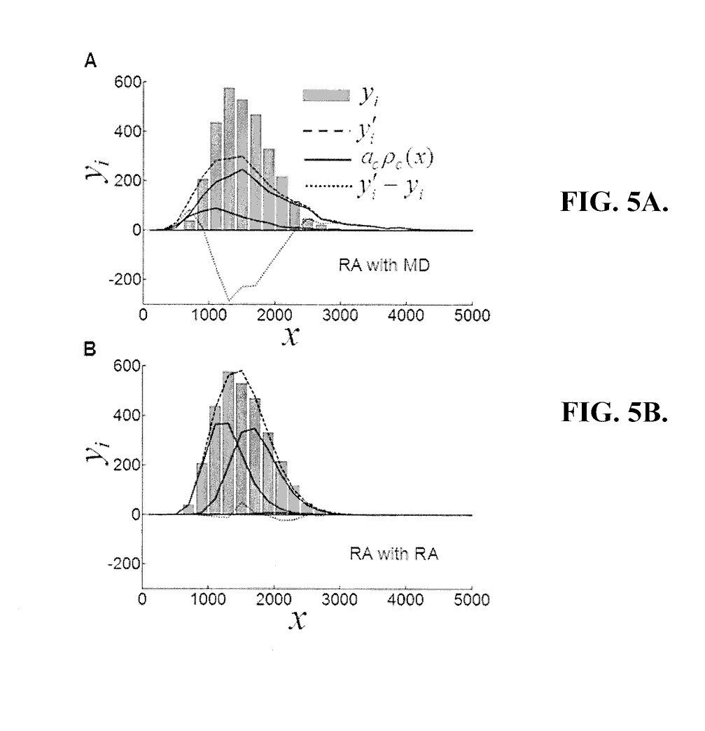

Case I. Table 2 lists the best-fit values of the coefficients a_{c}, N′ and χᵥ² for those simulations belonging to Case I (2RA, 3RA, 4RA, 5RA, 6RA and 8RA; see Table 1), which clearly indicates it is not possible to obtain a reasonable fit using MD basis vectors when the distribution is RA and has a relative width that is smaller than that of the single-molecule calibrating distribution. FIG. 5 illustrates an example, where the simulated distribution for 3RA is plotted along with the best-fit results obtained using both the MD and RA basis vectors.

TABLE 2

| | |
|---|---|
| | Simulated |
| Simulation | Distribution fit with |

| Simulation | | Value (b) | MD vectors | RA vectors |
|---|---|---|---|---|
| 2RA | a₂ | 1500 | 1417 | 1438 |
| | a₃ | 1500 | 1553 | 1511 |
| | N′ | — | 2970 | 3000 |
| | ΔN′ | — | 0 | 51 |
| | χᵥ² | — | 15.3 (c) | 0.12 |
| 3RA | a₃ | 1500 | 613 | 1428 |
| | a₄ | 1500 | 2255 | 1518 |
| | N′ | — | 2868 | 2995 |
| | ΔN′ | — | 0 | 49 |
| | χᵥ² | — | 66.2 (c) | 0.64 |
| 4RA | a₄ | 1500 | 0.02 | 1375 |
| | a₅ | 1500 | 2803 | 1580 |
| | N′ | — | 2803 | 2997 |
| | ΔN′ | — | 0 | 42 |
| | χᵥ² | — | 98.3 (c) | 0.48 |
| 5RA | a₂ | 3000 | 2944 | 2951 |
| | N′ | — | 2994 | 2997 |
| | ΔN′ | — | 0 | 46 |
| | χᵥ² | — | 27.6 (c) | 0.41 |
| 6RA | a₄ | 3000 | 1362 | 2852 |
| | N′ | — | 2567 | 3000 |
| | ΔN′ | — | 1205 | 48 |
| | χᵥ² | — | 215 (c) | 0.41 |
| 8RA | a₃ | 200 | 0.002 | 238 |
| | a₄ | 1200 | 876 | 1062 |
| | a₅ | 1200 | 2036 | 1243 |
| | a₆ | 400 | 0.13 | 448 |
| | N′ | — | 2912 | 2994 |
| | ΔN′ | — | 0 | 3 |
| | χᵥ² | — | 36.4 (c) | 0.59 |

Table 2 shows simulated intensity distributions and best-fit results for examples in Case I, where (σ/μ)_{distribution}<(σ/μ)₁. Each set of coefficients was used to generate intensity distributions using both the MD and RA process, and both resulting distributions were then fitted using both MD and RA basis vectors. The list of coefficients includes only those that had nonzero values in the simulated intensity distribution, and their sum is 3000 for all simulations. N′ is the sum of all best-fit coefficients. ΔN′ is the sum of the unlisted coefficients, that is, ΔN′ is the sum of those best-fit coefficients that would have been zero for a perfect fit. In some instances, the sum of the coefficients listed exceeds N′ due to rounding. Eight basis vectors were used for all fits listed in this table; α=0.001 unless otherwise stated. (b) Values of A_{c} used to generate the simulated distribution. (c) With α=0.001, this fit converged with to an N′ that differed from N=3000 by more than 1%; increasing ato 0.01 improved the agreement, but the resulting N′ were still not within 1% of N. Because increasing a further would increase the already very large value of χᵥ², the fits for larger values of a were not tested and the results shown are for a fit with α=0.01.

Case II. Unlike Case I, knowing that the relative width of the ROI distribution is larger than that of the single-molecule distribution does not provide an indication as to whether the fluorophore intensities were combined by a MD or RA process. Although measuring a lognormal intensity distribution for both single molecules and single clusters suggests strongly a MD process. For the examples in Case II, we will see that we can distinguish between the RA and MD process using the resultant goodness of fit.

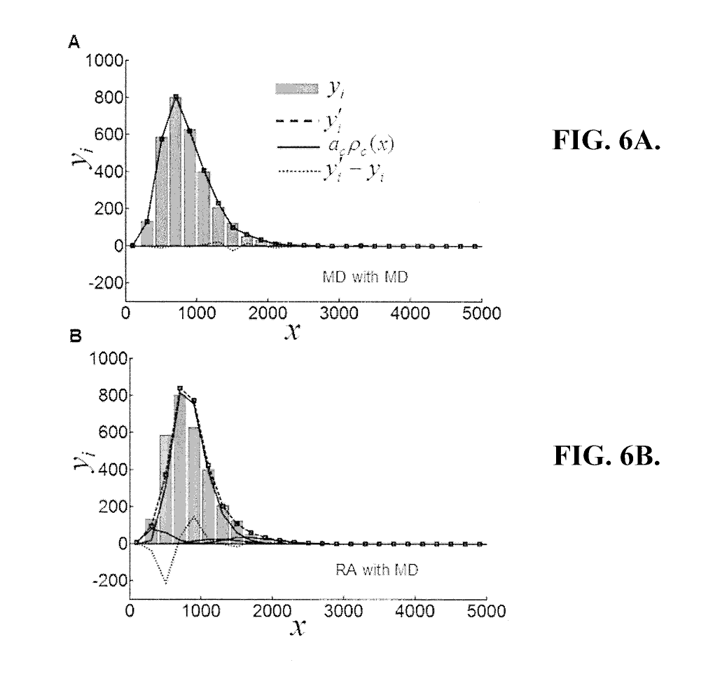

Table 3 lists the results of fits for Case II, which shows cases for which χᵥ² is consistently smaller when the distribution is fit with the correct basis vectors. This result indicates that for some cases if the shapes of the basis vectors are sufficiently different, then it is possible to distinguish between the MD and RA processes by comparing the χᵥ² of the resultant fits using the MD and RA basis vectors. FIG. 6 illustrates a Case II example, where the simulated distribution for 5MD is plotted along with the best-fit results using both the MD and RA basis vectors. The difference in χᵥ² listed in Table 3 is reflected in FIG. 6, where the best-fit result using the RA basis vectors cannot adequately describe the tails of the distribution generated from the MD basis vector.

TABLE 3

| | |
|---|---|
| | Simulated |
| Simulation | Distribution fit with |

| Simulation | | Value (b) | MD vectors | RA vectors |
|---|---|---|---|---|
| 1RA | a₁ | 1500 | 1376 | 1440 |
| | a₂ | 1500 | 1618 | 1552 |
| | N′ | — | 2994 | 2998 |
| | ΔN′ | — | 0 | 6 |
| | χᵥ² | — | 2.66 (c) | 0.29 |
| 7RA | a₁ | 200 | 86 | 191 |
| | a₂ | 1200 | 1038 | 1169 |
| | a₃ | 1200 | 1866 | 1219 |
| | a₄ | 400 | 0.002 | 418 |
| | N′ | — | 2990 | 2997 |
| | ΔN′ | — | 0 | 0 |
| | χᵥ² | — | 5.01 (c) | 0.25 |
| 2MD | a₂ | 1500 | 1390 | 1738 |
| | a₃ | 1500 | 1592 | 701 |
| | N′ | — | 2991 | 2986 |
| | ΔN′ | — | 9 | 547 |
| | χᵥ² | — | 0.65 | 1.88 |
| 5MD | a₂ | 3000 | 2993 | 2575 |
| | N′ | — | 2995 | 2994 |
| | ΔN′ | — | 2 | 419 |
| | χᵥ² | — | 0.34 | 5.62 (c) |

Table 3 lists simulated intensity distributions and best-fit results for examples in Case II, where (σ/μ)_{distribution}≧(σ/ρ)₁ but where there is a significant difference in χᵥ² between the MD and RA fits. Each set of coefficients was used to generate intensity distributions using both the MD and RA process, and both resultant distributions were then fit using both the MD and RA basis vectors. The list of coefficients includes only those that had nonzero values in the simulated intensity distribution, and their sum is 3000 for all simulations. N′ is the sum of all best coefficients. ΔN′ is the sum of the unlisted coefficients, that is, ΔN′ is the sum of those best-fit coefficients that would have been zero for a perfect fit. In some instances, the sum of the coefficients listed exceeds N′ due to rounding. Eight basis vectors were used for all fits listed in this table, and α=0.001, unless otherwise stated. (b) Values of A_{c} used to generate the simulate data. (c) With α=0.001, this fit converged to an N′ that differed from N=3000 by more than 1%. Increasing α to 0.01 improved the agreement, but the resulting N′ was still not within 1% of N. Because increasing further would increase the already very large value of χᵥ², the fits for larger values of α were not tested and the results shown are for a fit with α=0.01.

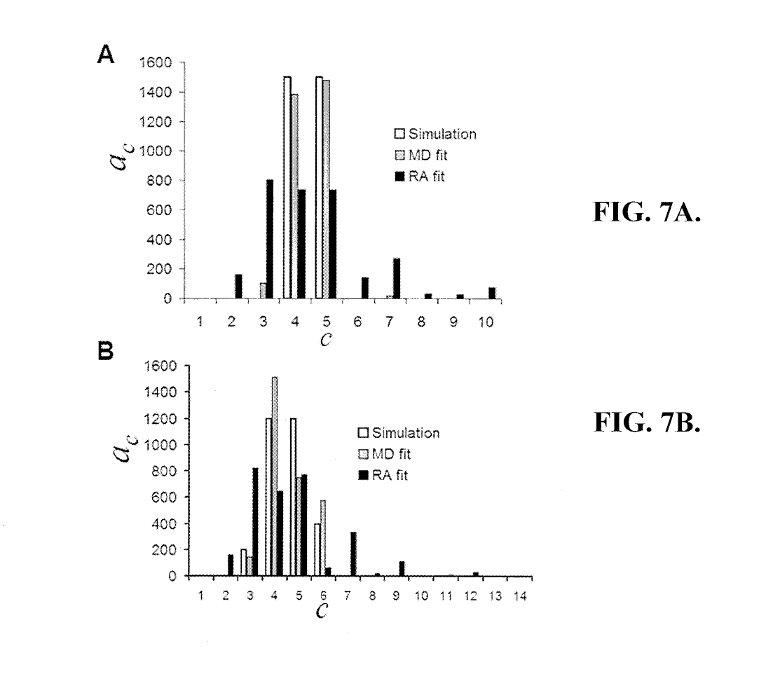

Case III. Table 4 lists the results for fits in Case III. All of these are examples of simulated distributions generated by the MD process, but for which modest and similar values of χᵥ² could be obtained in fits with either the RA or MD basis vectors. Two situations can result in this type of ambiguity. The first is illustrated by simulated distribution 1MD. Because f₁^{M}(i)≡f₁^{A}(i), it is not surprising that any distribution with a significant number of ROIs with c=1 (as is the case with 1MD) could be fit with either set of basis vectors and result in a modest and similar values of χᵥ². The second situation is illustrated by 4MD. While a modest value of χᵥ² can be obtained using RA basis vectors, it requires nonzero coefficients for 9 of the 10 RA basis vectors used in the fit, instead of the 2MD vectors used to generate it. FIG. 7A plots the coefficients used to generate 4MD, and the best-fit results using RA and MD basis vectors. The small differences between the AC used to generate the simulated distribution and the ac obtained from the fit with MD basis vectors are an illustration of the statistical errors inherent in the fitting process, and which should be kept in consideration once it is resolved whether MD or RA basis vectors are appropriate. Another example is provided by the fit to the 8MD simulated distribution. The 8MD distribution was generated using the MD process for c=3 to 6, and the fit with the MD basis vectors yields fair results. The vast majority of the ROIs are assigned to the correct four values of c, and the fit correctly orders a_{c} by size. The significant differences are an example of the effect of digitization noise; the binning of the simulated distribution functions results in sparsely populated bins at large intensities. Increasing the number of data points will reduce this error and improve the fit.

TABLE 4

| | |
|---|---|
| | Simulated |
| Simulation | Distribution fit with |

| Simulation | | Value (b) | MD vectors | RA vectors |
|---|---|---|---|---|
| 1MD | a₁ | 1500 | 1478 | 1721 |
| | a₂ | 1500 | 1515 | 1059 |
| | N′ | — | 2995 | 2994 |
| | ΔN′ | — | 2 | 214 |
| | χᵥ² | — | 0.34 | 0.74 |
| 3MD | a₃ | 1500 | 1291 | 1209 |
| | a₄ | 1500 | 1595 | 598 |
| | N′ | | 2992 | 2992 |
| | ΔN′ | | 6 | 1185 |
| | χᵥ² | | 0.55 | 0.89 |
| 4MD | a₄ | 1500 | 1385 | 738 |
| | a₅ | 1500 | 1482 | 736 |
| | N′ | — | 126 | 2993 |
| | ΔN′ | — | 2993 | 1519 |
| | χᵥ² | — | 0.47 | 0.73 (c) |
| 6MD | a₄ | 3000 | 2402 | 535 |
| | N′ | — | 2988 | 2973 |
| | ΔN′ | — | 586 | 2438 |
| | χᵥ² | — | 0.95 | 0.44 |
| 7MD | a₁ | 200 | 202 | 329 |
| | a₂ | 1200 | 1165 | 1354 |
| | a₃ | 1200 | 1278 | 795 |
| | a₄ | 400 | 287 | 299 |
| | N′ | — | 2992 | 2990 |
| | ΔN′ | — | 60 | 213 |
| | χᵥ² | — | 0.52 | 1.22 |
| 8MD | a₃ | 200 | 144 | 824 |
| | a₄ | 1200 | 1516 | 650 |
| | a₅ | 1200 | 753 | 773 |
| | a₆ | 400 | 579 | 66 |
| | N′ | — | 3000 | 3000 |
| | ΔN′ | — | 8 | 687 |
| | χᵥ² | — | 0.39 | 0.57 (d) |

Table 4 shows simulated intensity distributions and best-fit results for examples in Case III, where (σ/μ)_{distribution}≧(σ/μ), but there is no significant difference in χᵥ² between the MD and RA fits. Each set of coefficients was used to generate intensity distributions using both the MD and RA process, and both resultant distributions were then fit using both the MD and RA basis vectors. The list of coefficients includes only those that had nonzero values in the simulated intensity distribution, and their sum is 3000 for all simulations. N′ is the sum of all best coefficients. ΔN′ is the sum of the unlisted coefficients, that is, ΔN′ is the sum of those best-fit coefficients that would have been zero for a perfect fit. In some instances, the sum of the coefficients listed exceeds N′ due to rounding. Eight basis vectors were used for all fits listed in this table, and α=0.001, unless otherwise stated. (b) Values of A_{c} used to generate the simulated intensity distribution. (c) Ten RA basis vectors were used in this fit. (d) Fourteen RA basis vectors were used in this fit.

Resolving the ambiguity for Case III results requires either some a priori knowledge about the system or a method to calibrate the instrument and the experimental condition to verify independently whether MD or RA is the underlying process.

A priori knowledge. The fit with RA basis vectors would require contributions from a much wider range of c values than for the fit with MD basis vectors. In addition, the shape of the fitted distribution varies greatly when MD versus RA basis vectors is used. Therefore, if one knows there is a maximum number of fluorophores per ROI or if the shape (e.g., unimodal) of the ROI distribution is known, then often times this additional information may be sufficient to rule out the result obtained from using the incorrect basis vectors. The measurement of avidin-biotin binding discussed below is a good example, in which the fit using RA can be discarded because the result greatly exceeds the maximum number of binding sites present.

The shape of the resultant fits, in which distributions were simulated using a unimodal distribution of MD basis vectors with an average of 20 fluorophores per ROI. The fit with RA basis vectors yielded a multimodal distribution of RA vectors was explored. Here if it were known that the distribution of fluorophores should be unimodal, then the multimodal distribution from a fit using RA basis vectors could be rejected.

Calibration. If no a priori knowledge exists, then one needs to determine separately whether the instrument and experimental conditions would give rise to the RA or MD process. There are two approaches: (1) use a known system to calibrate the experiment; and (2) measure the ROI distribution for multiple unrelated systems. A calibrating system is described below that uses biotin-avidin binding, in which biotin binding was saturated to ensure there are four dye-tagged biotins per avidin. By taking single-biotin and single avidin/biotin measurements, then fitting the resultant distributions with MD and RA basis vectors, the correct basis vectors to use in subsequent experiments with unknown systems can be determined. The ROI distribution for multiple unrelated systems can also be measured. Here we rely on the fact that the presence of a lognormal distribution for the ROIs is a strong indication of the MD process and the presence of a normal distribution is a strong indication of the RA process. If the ROI distributions for multiple unrelated systems are all lognormal, that the MD basis vectors should be used. For example, one may measure the intensity distributions of fluorescent GFP clusters for different types of cells with different GFP-tagged proteins. If they all exhibit lognormal distribution, then MD is most likely the correct basis vectors.

Flow-based studies. In addition to counting molecules (single or groups) immobilized on a substrate, the method of the invention can be applied to the study of flow-based intensity bursts. Intensity distributions can be recorded from discrete burst events that correspond to single molecules or fluorescent puncta, which are composed of several molecules, flowing through a detection region. An intensity distribution for single molecules is used to calibrate the system and RA versus MD analysis is applied to count the numbers of molecules bound to fluorescent puncta in flow. The distribution used for calibration is referred to as the single-molecule distribution or single-fluorophore distribution.

To demonstrate the method of the invention and to devise a calibration system, the number of fluorescently labeled biotin molecules bound to a single avidin protein was measured. Avidin binds biotin with a stoichiometry of 1:4, and the affinity is one of the strongest known interactions between a protein and a ligand. To ensure a 1:4 binding ratio, avidin was incubated with excess biocytin-Alexa 488 overnight after which unbound biocytin was subsequently removed using size-exclusion chromatography.

Avidin having one bound biocytin was used as the single-molecule calibrating intensity distribution (i.e., as the c=1 basis vector), which takes into account any fluorescence quenching that may have occurred when Alexa-tagged biocytin is bound to avidin. There is, however, self-quenching of the fluorophores when four Alexa-biocytin are bound to a single avidin molecule. To account for this self-quenching, a bulk assay was used to estimate the average number of Alexa-biocytin bound to avidin, and an average value of 3.7 biocytin per avidin was determined. Because the average value obtained using the single-molecule distributions was 2 rather than 3.7, a correction factor of 0.54 was used. This correction factor was then applied to the basis vectors to account for the presence of dye-dye self-quenching.

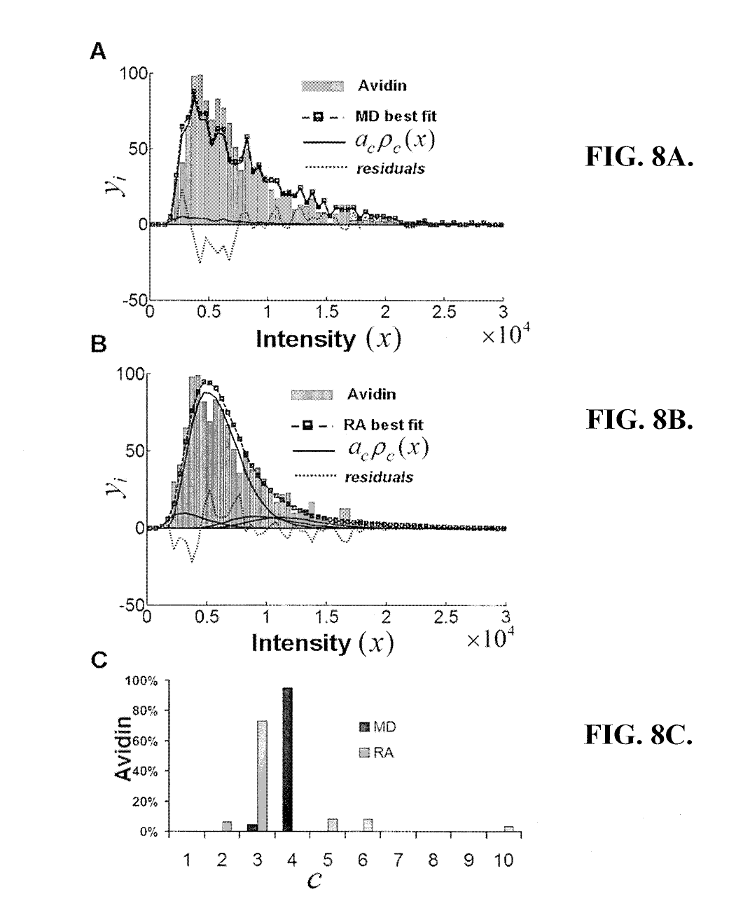

FIG. 8 shows the results of the analysis. A lognormal intensity distribution for the avidin molecules was measured, which implies a MD process. FIG. 8A shows the result of the fitting using MD basis vectors, which resulted in 95% four and 5% three biocytin molecules per avidin, with a reduced chi squared value of 1.18 and a non-physical parameter of 0%. The non-physical parameter is the percentage of avidin molecules that have more than four bound biocytin. FIG. 8B shows the result of the fit using RA basis vectors, which resulted in 6.3% two, 73% three, 8.3% five, 8.3% six and 4% ten biocytin per avidin, with a reduced chi squared value of 1.49 and a non-physical parameter of 20.6%. This avidin-biocytin binding may serve as one suitable calibration system for TIRF microscopy.

While illustrative embodiments have been illustrated and described, it will be appreciated that various changes can be made therein without departing from the spirit and scope of the invention.

1. A method for quantifying fluorescent puncta, comprising:
(a) acquiring at least one first intensity distribution, wherein the first intensity distribution comprises fluorescence intensity values from a plurality of first fluorescent puncta, and wherein each first fluorescent puncta comprises at least one fluorescent emitter;
(b) acquiring at least one second intensity distribution, wherein the second intensity distribution comprises fluorescence intensity values from a plurality of second fluorescent puncta, wherein each second fluorescent puncta has a determined number of fluorescent emitters;
(c) determining the relationship between the first and second intensity distributions; and
(d) fitting the second intensity distribution to the first intensity distribution to provide a count and distribution of the number of fluorescent emitters within the first fluorescent puncta.

2. The method of claim 1, wherein the first intensity distribution is a normal distribution, a lognormal distribution, or a combination thereof.

3. The method of claim 1, wherein the second intensity distribution is a normal distribution, a lognormal distribution, or a combination thereof.

4. The method of claim 1, wherein determining the relationship between the first and second intensity distributions comprises scale analysis.

5. The method of claim 1, wherein determining the relationship between the first and second intensity distributions comprises shape analysis.

6. The method of claim 1, wherein determining the relationship between the first and second intensity distributions comprises statistical analysis.

7. The method of claim 1, wherein determining the relationship between the first and second intensity distributions comprises analytical calibration analysis.

8. The method of claim 1, wherein the relationship between the first and second intensity distributions is multiplicative, additive, or a combination thereof.

9. The method of claim 1, wherein fitting the second intensity distribution to the first intensity distribution comprises a closeness-of-fit calculation.

10. The method of claim 1, wherein the fluorescent puncta comprise at least one fluorescent emitter.

11. The method of claim 1, wherein the fluorescent emitter is an emitter selected from the group consisting of a molecule, an antibody, an antibody fragment, a protein, a peptide, a subcellular organelle, a subcellular structure, a subcellular compartment, a protein complex, a nanoparticle, and a quantum dot.

12. The method of claim 1, wherein the fluorescent puncta comprise at least one fluorescent emitter in contact with at least one synaptic vesicle, liposome, biological cell, organelle, protein, DNA, signaling complex, or antibody.

13. The method of claim 1, wherein the fluorescent puncta are in a cell, on a cell, immobilized, or in flow.

14. The method of claim 1, wherein the fluorescent puncta result from single photon excitation, two-photon excitation, multi-photon excitation, chemiluminescent excitation, or electroluminescent excitation.

15. The method of claim 1, wherein the intensity values are acquired by an imaging system selected from the group consisting of a wide area epifluorescence microscope, a confocal microscope, a scanning confocal microscope, a total internal reflection fluorescence microscope, a two-photon microscope, a scanning two-photon microscope, and a stimulated emission depletion microscope.
